(19) United States
(12) Patent Application Publication
(10) Pub. No.: US 2015/0157864 A1
(43) Pub. Date: Jun. 11, 2015

(54) APPARATUS, METHOD AND SYSTEM FOR NOISE STIMULATION

(71) Applicant: Pacesetter, Inc., Sylmar, CA (US)

(72) Inventor: Stuart Rosenberg, Castaic, CA (US)

(21) Appl. No.: 14/562,539

(22) Filed: Dec. 5, 2014

Related U.S. Application Data
(60) Provisional application No. 61/912,422, filed on Dec. 5, 2013.

(51) Int. Cl. A61N 1/36

(52) U.S. Cl. CPC A61N 1/36139; A61N 1/36171; A61N 1/36175; A61N 1/36157

(57) ABSTRACT

The present disclosure provides systems and methods utilizing a noise stimulation apparatus. The apparatus includes at least one stimulating electrode and an implantable pulse generator coupled to the at least one stimulating electrode. The implantable pulse generator includes an accelerometer and a computing device coupled to the accelerometer. The computing device is configured to cause the implantable pulse generator to generate at least one stimulation pulse based on signals received from the accelerometer.

### CROSS REFERENCE TO RELATED APPLICATIONS

This application claims the benefit of U.S. Provisional Application Ser. No. 61/912,422, filed Dec. 5, 2013.

### FIELD OF THE DISCLOSURE

The present disclosure relates generally to neurostimulation methods and systems, and more particularly to an apparatus that applies noise stimulation using signals from an accelerometer.

### BACKGROUND ART

Neurostimulation is a treatment method utilized for managing the disabilities associated with pain, movement disorders such as Parkinson's Disease (PD), dystonia, and essential tremor, and also a number of psychological disorders such as depression, mood, anxiety, addiction, and obsessive compulsive disorders. In addition to conventional tonic stimulation and burst stimulation, other stimulation paradigms exist. One such stimulation paradigm is “noise” stimulation, wherein a current or voltage controlled signal is driven with noise of a particular color, such as white noise (i.e., equal power in all frequency bands) or pink noise (i.e., greater power in lower frequency bands).

Notably, noise stimulation may be more effective for treating certain conditions, such as tinnitus and pain. Further, noise stimulation may avoid progressive loss of efficacy due to the random nature of the stimulation, and may also activate different subpopulations of neurons due to the spectrum of frequencies delivered. Accordingly, it would be desirable to have a neurostimulation apparatus capable of delivering true noise stimulation to a subject, along with related systems and methods of use.

### BRIEF SUMMARY OF THE DISCLOSURE

In one embodiment, the present disclosure is directed to a noise stimulation apparatus for generating electrical pulses for application to tissue of a patient. The noise stimulation apparatus includes at least one stimulating electrode adapted to apply stimulation pulses to tissue of the patient and an implantable pulse generator coupled to the at least one stimulating electrode. The implantable pulse generator includes an accelerometer and a computing device coupled to the accelerometer. The computing device is configured to cause the implantable pulse generator to generate at least one stimulation pulse based on signals received from the accelerometer.

In another embodiment, the present disclosure is directed to an implantable pulse generator for generating electrical pulses for application to tissue of a patient. The implantable pulse generator includes an accelerometer configured to measure accelerations experienced by the accelerometer and generate signals indicative of the measured accelerations. The implantable pulse generator further includes a computing device coupled to the accelerometer. The computing device is configured to receive the signals from the accelerometer, map the received signals to at least one output parameter, and cause the implantable pulse generator to generate at least one stimulation pulse having the at least one output parameter.

In another embodiment, the present disclosure is directed to a method for applying noise stimulation to a subject. The method includes measuring accelerations using an accelerometer, generating, using the accelerometer, signals indicative of the measured accelerations, and generating, using an implantable pulse generator, at least one stimulation pulse based on the generated signals.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIGS. 2A-2C are schematic views of stimulation portions that may be used with stimulation system of FIG. 1.

FIG. 3 is a schematic view of one embodiment of a noise stimulation apparatus including at least one stimulating electrode and an implantable pulse generating having an accelerometer.

FIG. 4 is a block diagram of one embodiment of a computing device that may be used with the noise stimulation apparatus of FIG. 3.

FIG. 5 is a flow chart of one embodiment of a method for applying noise stimulation to a subject.

Corresponding reference characters indicate corresponding parts throughout the several views of the drawings.

### DETAILED DESCRIPTION OF THE DISCLOSURE

The present disclosure provides systems and methods utilizing a noise stimulating apparatus. The noise stimulating apparatus includes at least one stimulating electrode coupled to an implantable pulse generator. The implantable pulse generator includes an accelerometer, and generates at least one stimulation pulse based on accelerations measured by the accelerometer. The at least one stimulation pulse is delivered to a subject via the at least one stimulating electrode.

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

Peripheral nerve field stimulation (PNFS) is another form of neuromodulation. The basic devices employed for PNFS are similar to the devices employed for SCS including pulse generators and stimulation leads. In PNFS, the stimulation leads are placed in subcutaneous tissue (hypodermis) in the area in which the patient experiences pain. Electrical stimulation is applied to nerve fibers in the painful area. PNFS has been suggested as a therapy for a variety of conditions such as migraine, occipital neuralgia, trigeminal neuralgia, lower back pain, chronic abdominal pain, chronic pain in the extremities, and other conditions.

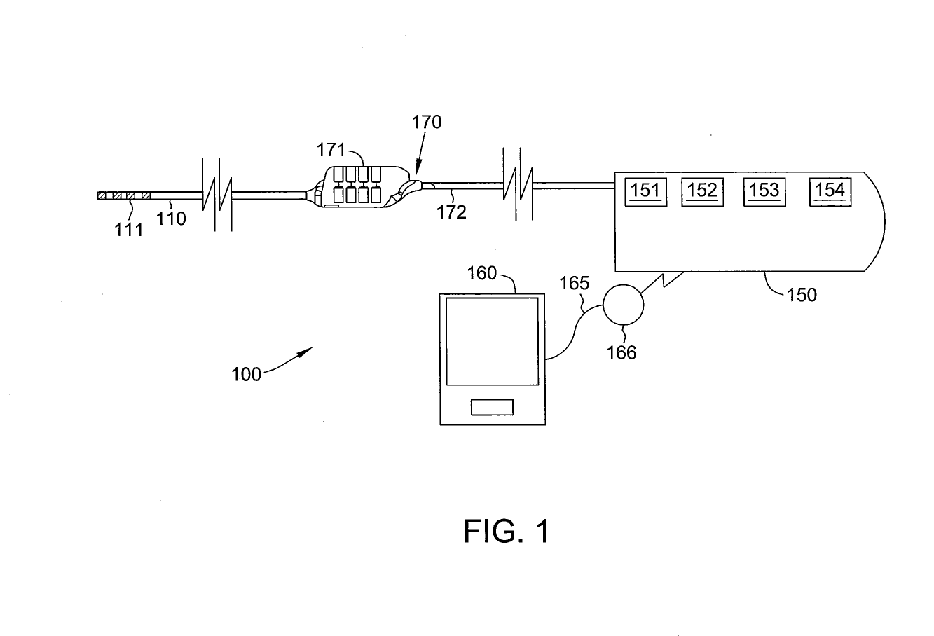

Referring now to the drawings, and in particular to FIG. 1, a stimulation system is indicated generally at 100. Stimulation system 100 generates electrical pulses for application to tissue of a patient, or subject, according to one embodiment. System 100 includes an implantable pulse generator 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of pulse generator 150 for execution by the microcontroller or processor to control the various components of the device.

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

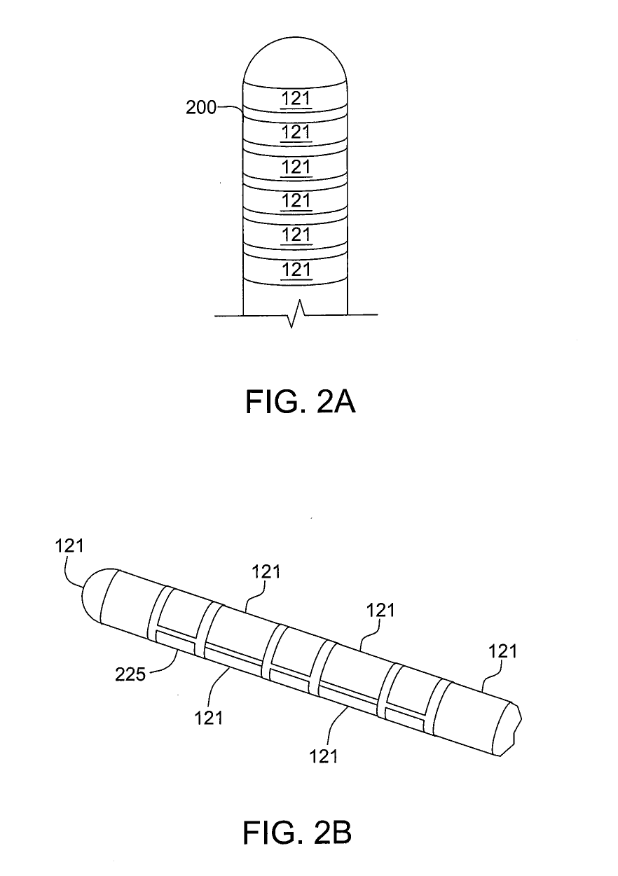

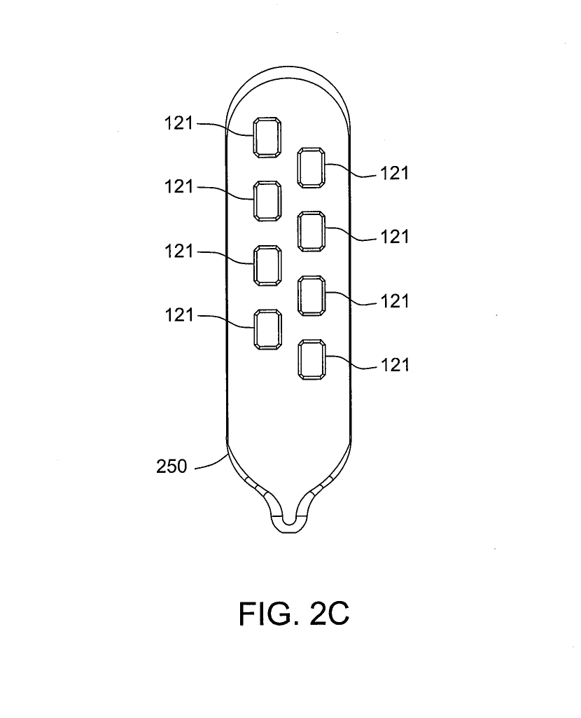

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2010072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Controller device 160 may be implemented to recharge battery 153 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of generator 150. The charging circuitry may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical, Inc. (Plano, Tex.). Example commercially available stimulation leads include the QUATTRODE™, OCTRODE™, AXXESS™, LAMITRODE™, TRIPOLE™, EXCLAIM™, and PENTA™ stimulation leads from St. Jude Medical, Inc.

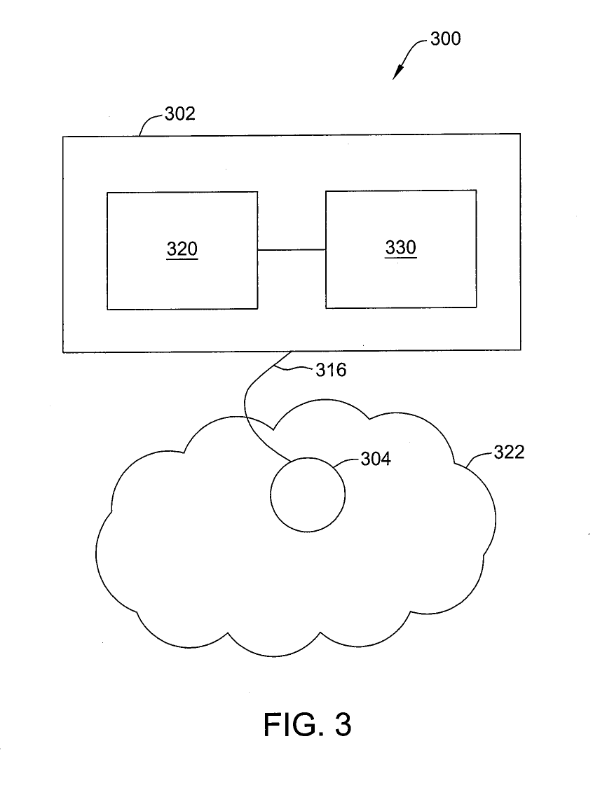

In FIG. 3, an implantable noise stimulation apparatus is indicated generally at 300. Apparatus 300 includes an implantable pulse generator (IPG) 302 electrically coupled to at least one stimulating electrode 304. IPG 302 may be implanted, for example, on the skull of a subject, or in any other suitable body area. In the illustrated embodiment, apparatus 300 includes one stimulating electrode 304. Alternatively, in other suitable embodiments, apparatus 100 may include a plurality of stimulating electrodes 304.

Stimulating electrode 304 is implanted in a region 306 of the subject's nervous system and is electrically coupled to IPG 302 by a conductive lead 308. For example, stimulating electrode 304 may be implanted in a subdural surface, an epidural surface, a subcutaneous peripheral nerve field, and/or a subcortical structure. Stimulating electrode 304 applies noise stimulation to neurons located in region 306, as described in detail herein.

In the illustrated embodiment, IPG 302 includes an accelerometer 320 communicatively coupled to a computing device 330. IPG 302 generates and supplies stimulation pulses to stimulating electrode 304 based at least in part on accelerations measured by accelerometer 320. In the illustrated embodiment, accelerometer 320 is a three-dimensional accelerometer configured to measure accelerations in an x-direction, a y-direction orthogonal to the x-direction, and a z-direction orthogonal to both the x- and y-directions. For example, accelerometer 320 may be a micro-electro-mechanical system (MEMS) accelerometer. Alternatively, accelerometer 320 may be any acceleration-measuring device that enables apparatus 300 to function as described herein. Other accelerometers are also within the scope of the present disclosure.

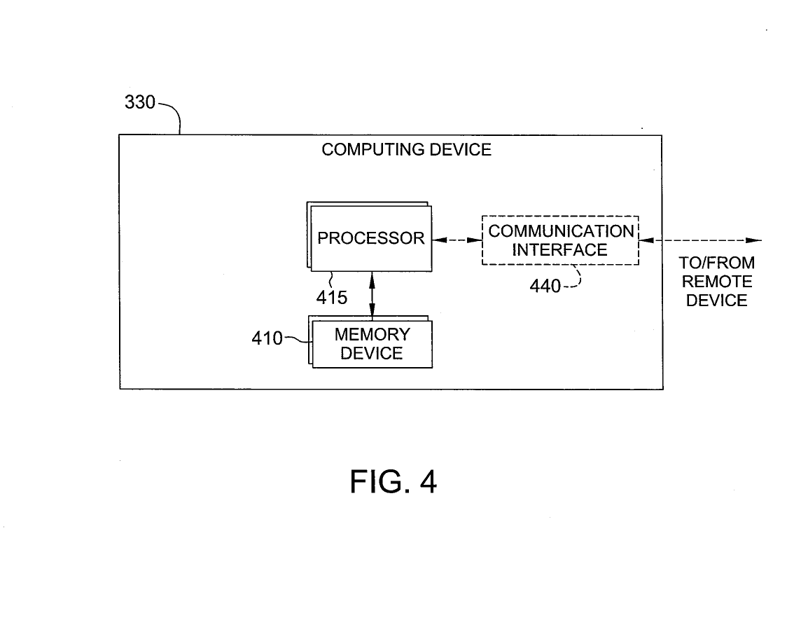

FIG. 4 is a block diagram of one embodiment of a suitable computing device 330 that may be used with IPG 302 (shown in FIG. 1). Computing device 330 includes at least one memory device 410 and a processor 415 that is coupled to memory device 410 for executing instructions. In some embodiments, executable instructions are stored in memory device 410. In the illustrated embodiment, computing device 330, and by extension IPG 302, performs one or more operations described herein by programming processor 415. For example, processor 415 may be programmed by encoding an operation as one or more executable instructions and by providing the executable instructions in memory device 410.

Processor 415 may in some suitable embodiments include one or more processing units (e.g., in a multi-core configuration). Further, processor 415 may be implemented using one or more heterogeneous processor systems in which a main processor is present with secondary processors on a single chip. In another illustrative example, processor 415 may be a symmetric multi-processor system containing multiple processors of the same type. Further, processor 415 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein. In the illustrated embodiment, processor 415 processes signals received from accelerometer 320 and controls IPG 302 to deliver one or more pulses to stimulating electrode 304 based at least in part on the received signals, as described herein.

In the illustrated embodiment, memory device 410 is one or more devices that enable information such as executable instructions and/or other data to be stored and retrieved. Memory device 410 may include one or more computer readable media, such as, without limitation, dynamic random access memory (DRAM), static random access memory (SRAM), a solid state disk, and/or a hard disk. Memory device 410 may be configured to store, without limitation, application source code, application object code, source code portions of interest, object code portions of interest, configuration data, execution events and/or any other type of data.

Computing device 330, in the illustrated embodiment, includes a communication interface 440 coupled to processor 415. Communication interface 440 communicates with one or more remote devices, such as a clinician or patient programmer (not shown). To communicate with remote devices, communication interface 440 may include, for example, a wired network adapter, a wireless network adapter, a radio-frequency (RF) adapter, and/or a mobile telecommunications adapter.

IPG 302 utilizes accelerometer 320 to generate pseudorandom stimulation pulses, as described herein. More specifically, when implanted in a subject, accelerometer 320 is subjected to relatively random accelerations (e.g., during motion of the subject). Further, even when subject is relatively still, at high gain, accelerometer 320 may detect accelerations due to vibrations from respiration, blood pressure pulse, ambient acoustic noise, etc. Accelerometer 320 transmits signals indicative of measured accelerations to computing device 330, and those signals are mapped to generate electrical stimulation pulses, as described herein. In general, the apparatus may map signals from accelerometer 320 into stimulation pulses using either frequency mapping or bit mapping. As the accelerations measured by accelerometer 320 are substantially random, the stimulation pulses generated from the mapping are also substantially random (i.e., “noisy”). IPG 302 may be a current-controlled pulse generator that generates current pulses, or a voltage-controlled pulse generator that generates voltage pulses. Accordingly, as used here, “stimulation pulses” refers to current pulses and/or voltage pulses.

In some embodiments, the signals from accelerometer 320 are mapped to output parameters (e.g., amplitude, frequency, pulse width) for the stimulation pulses. By mapping the accelerometer signals to output parameters, pulses (e.g., square waves) are delivered at seemingly random intervals and random energy, which will be interpreted by the nervous system as noise. Further, the mapping may be controlled to produce different colors of noise stimulation. Multiple techniques having different degrees of complexity and generating stimulation having different degrees of randomness are described herein. However, those of skill in the art will appreciate that other techniques not specifically described herein are within the spirit and scope of the disclosure. Notably, the systems and methods described herein may be utilized in a wide variety of neurostimulation applications. For example, the embodiments described herein may be used in spinal cord stimulation (SCS) to facilitate treating chronic pain, or in cortical stimulation to facilitate treating, for example, tinnitus or central pain.

In some embodiments, signals from accelerometer 320 are mapped to stimulation pulses using frequency mapping. More specifically, signals from accelerometer 320 are filtered, for example, using processor 415 or separate filtering circuitry, into a passband that covers a range of frequencies at which IPG 302 is able to generate stimulation pulses. For example, signals from accelerometer 320 may be filtered into a passband ranging from approximately 2 Hertz (Hz) to 1200 Hz. Signals may also be filtered in embodiments that utilize bit mapping instead of frequency mapping.

For frequency mapping, using an appropriate sampling window, a power spectrum for the filtered signals is calculated, using, for example, processor 415. For example, the filtered signals may be sampled at approximately 2 kilohertz (kHz), or at other suitable rates. The power at each frequency is then scaled to a current output value, and IPG 302 generates stimulation pulses having the corresponding frequency and current output value. For example, the power may be scaled to a current output value in a range of from about 0.1 milliamps (mA) to about 20.0 mA. Alternatively, the power may be scaled to any range of current output values that enables IPG 302 to function as described herein. Accordingly, for frequency mapping, signals from accelerometer 320 are filtered, a power spectrum is calculated for the filtered signals, the calculated power values are scaled to corresponding current output values, and stimulation pulses having the frequency and associated current are generated by IPG 302, resulting in substantially random (i.e., “noisy”) stimulation of neurons in region 306.

In some embodiments, signals from accelerometer 320 are mapped to stimulation pulses using bit mapping. For bit mapping, signals from accelerometer 320 are digitized using an at least 8-bit analog to digital (A/D) converter. The digitized bits are then mapped to output parameters of the stimulation pulses generated by IPG 302.

In the illustrated embodiment, as explained above, accelerometer 320 is a three-dimensional accelerometer. Accordingly, accelerometer 320 measures an x-component of an acceleration experienced by accelerometer 320, a y-component of the acceleration, and a z-component of the acceleration. In some embodiments, each of the x-, y-, and z-components are mapped to one output parameter (e.g., amplitude, frequency, pulse width) of the stimulation pulses.

In one embodiment, the x-component is mapped to pulse frequency, the y-component is mapped to pulse width, and the z-component is mapped to pulse amplitude. For example, assume accelerometer 320 measures an acceleration having an x-component of 1.0 milli-g (mG), where g is acceleration due to gravity, a y-component of 1.5 mG, and a z-component of 0.3 mG. These measurements are digitized into 1010,0000 for the x-component, 1110,1000 for the y-component, and 0010,0011 for the z-component. For the next stimulation pulse generated by IPG 302, registers or locations in memory device 410 representing pulse frequency are programmed to 1010,0000, registers or locations representing pulse width are programmed to 1110,1000, and registers representing pulse amplitude are programmed to 0010,0011. This may result, for example, in IPG 302 generating stimulation pulses having an output frequency of 530 Hz, a pulse width of 875 microseconds (μs), and a pulse amplitude of 2.4 milliamps (mA). Notably, the process of digitizing signals from accelerometer 320, programming stimulation output parameter registers, and generating and delivering stimulation pulses may be repeated as long as stimulation as desired.

As will be appreciated by those of skill in the art, the actual mapping between digitized accelerometer bits and output parameters may be adjusted or customized for the subject based on a plurality of profiles stored, for example, in memory device 410. In one example, ranges of the pulse frequency, pulse width, and pulse amplitude may be set to match ranges of known efficacy or comfort for the subject. In another example, the accelerometer signals may be passed through an auto-gain algorithm to result in a wider range of output parameters, which may facilitate achieving a full spectrum of noise simulation. In yet another example, the mapping might utilize a transfer function that weighs lower frequencies more heavily than higher frequency, generating red or pink noise (i.e., noise with a frequency spectrum weighted heavier for lower frequencies) instead of white noise (i.e., noise with an equally weighted frequency). Further, in yet another example, the mapping might utilize a transfer function that generates a gray noise frequency spectrum (i.e., a frequency spectrum that is weighted but appears to be equal at all frequencies).

In some embodiments, the randomness of the generated stimulation pulses may be enhanced using techniques in addition to mapping signals from accelerometer 320. In one embodiment, the mapping function between the accelerometer signals and the output parameters changes over time or with subsequent stimulation pulse sequences. For example, the digitized bits from the accelerometer signals may be shifted by one position for each subsequent stimulation pulse delivered, before applying the mapping to output parameters.

In another embodiment, a device clock of, for example, IPG 302, is used to modify the mapping. For example, the least significant bits (LSB) of the device clock may be used as an AND, OR, XOR, NOR, etc., mask before the accelerometer signals are mapped to output parameters.

The device clock functions as a digital counter that counts “ticks” from some predetermined start time (e.g., a time processor 415 was first powered on, or a time firmware was loaded). Ticks are a relatively small unit of time relevant to processor 415 or a multiple thereof. For example, one tick may be 50 milliseconds (ms). As used herein, the LSB are the bits corresponding to the smallest time interval. For example, if the device clock has 24 bits, the bitmask may utilize the last (i.e., rightmost) 8 bits. If one tick corresponds to 50 ms, the 8 LSB correspond to a time from 0 to 12,800 ms. Accordingly, a bit mask using the 8 LSB would recycle once approximately every 13 seconds.

Returning to the above example, suppose the measurements from accelerometer 320 are digitized into 1010,0000 for the x-component, 1110,1000 for the y-component, and 0010,0011 for the z-component. Also, suppose the 8 LSB on the device clock are 1010,1111. Using, for example, an XOR mask, the bits programmed onto pulse frequency registers are XOR(x;clock)=XOR(1010,0000; 1010,1111)=0000,1111, the bits programmed onto the pulse width registers are XOR(y;clock)=XOR(1110,1000; 1010,1111)=0100,0111, and the bits programmed onto the pulse amplitude registers are XOR(z;clock)=XOR(0010,0011; 1010,1111)=1000,1100. In this embodiment, the clock bits increment and are sampled at an interval more frequent that each and every increment, such that the mask does not count up in a linear manner. Such bit masking adds randomness, particularly when accelerometer 320 is subject to periodic motion.

Further, the choice of logic operator utilized in the bit masking can be tailored to add “color” to the noise generated. For example, logic operators for which truth tables have 3 FALSE values and 1 TRUE value (e.g., NOR, for which only a value of 0 for accelerometer 302 and a value of 0 for the clock will have a result of 1 to be programmed) will tend to result in the output bit values more likely being 0 instead of 1. When these bit values are mapped onto registers that encode a programmed stimulation frequency, the resultant frequency will be weighted towards lower frequencies in the range. While this result may not be strictly “pink” noise, it will approximate “pink” noise sufficiently well to achieve a desirable physiological outcome.

In yet another embodiment, to enhance randomness, a randomness extractor algorithm programmed on, for example, processor 415, may be utilized to extract non-random data from the accelerometer bit stream. The randomness extractor algorithm may be, for example, a 160-bit Secure Hash Algorithm (SHA).

Although a number of techniques for mapping accelerometer signals to stimulation pulses are described herein, those of skill in the art will appreciate that additional techniques are within the spirit and scope of this disclosure. For example, in one embodiment, instead of mapping accelerometer signals to output parameters, the accelerometer signals are used to generate a seed for a lookup table. Specifically, in this embodiment, a three-dimensional look up table is stored in, for example, memory device 410, and each cell in the lookup table includes predefined values for the output parameters. The x-component, y-component, and z-component are used to identify a corresponding cell in the lookup table, and the predefined values in the identified cell are used by IPG 302 to generate the stimulation pulses.

In the exemplary embodiment, a charge balancing technique is utilized to control the total amount of current delivered to the subject via the stimulation pulses. That is, too much current may damage IPG 302 or stimulating electrode 304, or may injure the subject. Accordingly, charge balancing ensures that any built-up charge is safely discharged. Charge balancing may be performed after each stimulation pulse delivered by stimulating electrode 304 or after a group of pulses that in aggregate sum to a predetermined charge delivery.

When the noise simulation delivered is pink noise, red noise, or otherwise weighted with higher power at lower frequencies, there will be a relatively high probability of periods when stimulation is not applied. During these periods, active charge balancing is used to actively discharge, or bleed off, stored charge. Active charge balancing may be accomplished, for example, by reversing a polarity of connections to a stimulation output capacitor, or by utilizing switches and/or transistors to drive built up charge from the simulation output capacitor to ground. Accordingly, at low frequencies, active charge balancing will not interrupt or otherwise interfere with the applied noise stimulation. Internal circuitry and algorithms for controlling the use of active and passive discharge operations in an implantable pulse generator are described in U.S. Pat. No. 8,095,221, which is incorporated by reference.

When noise stimulation and active charge balancing are not being utilized, passive charge balancing may be used or charge balancing may be omitted, allowing charge to build up. Passive charge balancing may include, for example, a relatively high-impedance resistor positioned between the stimulation output capacitor and ground. In this configuration, whenever stimulation and active charge balancing are not being performed, any charge built up on the stimulation output capacitor will slowly bleed to ground with a current smaller than that required to cause excitation of tissue in the vicinity of simulation electrode 304.

For embodiments without passive charge balancing, processor 415 may include controls that prevent stimulation programs that could lead to relatively high charge build up on stimulation output capacitors. For example, processor 415 may use logic to limit certain combinations of stimulation parameters for which active charge balancing would not be possible.

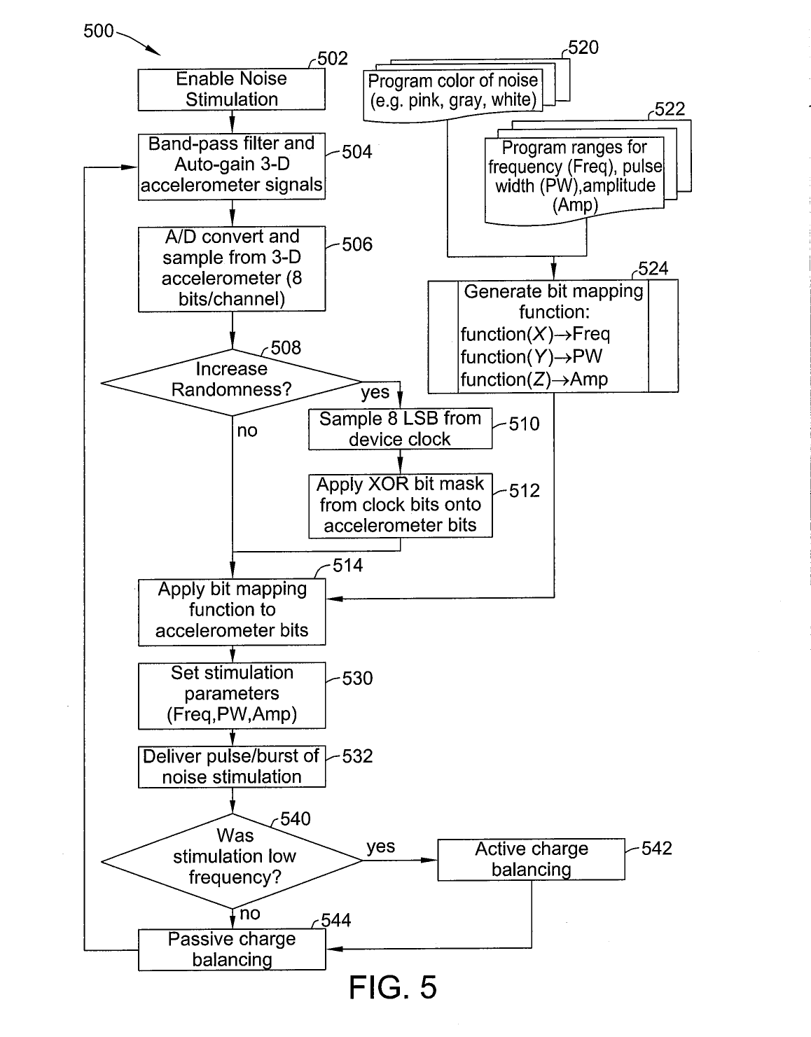

FIG. 5 is a flow chart of one embodiment of a method 500 for applying noise stimulation to a subject. In the embodiment illustrated in FIG. 5, noise stimulation is applied using bit mapping. However, as described above, noise stimulation could alternatively be applied using frequency mapping.

At block 502, a determination is made to enable noise stimulation using, for example, apparatus 300 (shown in FIG. 3). At block 504, signals received from an accelerometer, such as accelerometer 320 (shown in FIG. 3) are passed through a filter and an auto-gain algorithm using, for example, a processor, such as processor 415 (shown in FIG. 4). At block 506 the analog signals are converted into digital bits.

At block 508, it is determined whether the randomness of the noise stimulation should be increased. In the illustrated embodiment, if the randomness is increased, it is increased by sampling eight least significant bits from a device clock at block 510, and applying a XOR bit mask from the clock bits onto the accelerometer bits at block 512. Alternatively, different techniques for increasing the randomness, such as those described above, may be utilized. If the randomness is not to be increased, flow proceeds directly to block 514, at which a bit mapping function is used (e.g., by the processor) to map the accelerometer bits to stimulation output parameters.

The particular bit mapping function is determined by programming the processor. For example, in the illustrated embodiment, the desired type of noise is programmed at block 520, ranges for the output parameters are programmed at block 522, and the bit mapping function itself (mapping x-, y-, and z-components of acceleration to respective output parameters) is generated at block 524. The processor may be programmed wirelessly using, for example, a clinician programmer or a patient programmer communicatively coupled to computing device 330 (shown in FIGS. 3 and 4).

Based on the mapping performed at block 514, the stimulation parameters are set at block 530. An IPG, such as IPG 302 (shown in FIG. 3), generates stimulation pulses based on the parameters set at block 530, and the stimulation pulses are delivered via a stimulating electrode, such as stimulating electrode 304 (shown in FIG. 3), at block 532.

At block 540, the processor determines whether the applied stimulation was low frequency stimulation. If so, active charge balancing is applied at block 542. If not, only passive charge balancing is performed at block 544. Flow then returns to block 504 to continue providing stimulation.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A noise stimulation apparatus for generating electrical pulses for application to tissue of a patient, the noise stimulation apparatus comprising:
at least one stimulating electrode adapted to apply stimulation pulses to tissue of the patient; and
an implantable pulse generator coupled to the at least one stimulating electrode and comprising:
an accelerometer; and
a computing device coupled to the accelerometer, the computing device configured to cause the implantable pulse generator to generate at least one stimulation pulse based on signals received from the accelerometer.

2. The noise stimulation apparatus of claim 1 wherein the computing device is configured to map the signals received from the accelerometer to at least one output parameters of the at least one stimulation pulse.

3. The noise stimulation apparatus of claim 2 wherein the accelerometer is a three-dimensional accelerometer configured to measure accelerations in a first direction, a second direction orthogonal to the first direction, and a third direction orthogonal to the first and second directions.

4. The noise stimulation apparatus of claim 2 wherein the computing device is configured to:
map acceleration in the first direction to a frequency of the at least one stimulation pulse;
map acceleration in the second direction to a pulse width of the at least one stimulation pulse; and
map acceleration in the third direction to an amplitude of the at least one stimulation pulse.

5. The noise stimulation apparatus of claim 2 wherein the computing device is configured to:
filter the signals received from the accelerometer;
sample the filtered signals to generate a power spectrum; and
scale power values in the power spectrum to current values for the at least one stimulation pulse.

6. The noise stimulation apparatus of claim 1 wherein the computing device is configured to increase a randomness of the at least one stimulation pulse using least significant bits of a device clock of the implantable pulse generator.

7. The noise stimulation apparatus of claim 1 wherein the computing device is configured to apply charge balancing to limit a total amount of current delivered via the at least one stimulating electrode.

8. An implantable pulse generator for generating electrical pulses for application to tissue of a patient, the implantable pulse generator comprising:
an accelerometer configured to:
measure accelerations experienced by the accelerometer; and
generate signals indicative of the measured accelerations; and
a computing device coupled to the accelerometer, the computing device configured to:
receive the signals from the accelerometer;
map the received signals to at least one output parameter; and
cause the implantable pulse generator to generate at least one stimulation pulse having the at least one output parameter.

9. The implantable pulse generator of claim 8 wherein the accelerometer is a three-dimensional accelerometer configured to measure accelerations in a first direction, a second direction orthogonal to the first direction, and a third direction orthogonal to the first and second directions.

10. The implantable pulse generator of claim 9 wherein the computing device is configured to:
map acceleration in the first direction to a frequency of the at least one stimulation pulse;
map acceleration in the second direction to a pulse width of the at least one stimulation pulse; and
map acceleration in the third direction to an amplitude of the at least one stimulation pulse.

11. The implantable pulse generator of claim 8 wherein the computing device is configured to:
filter the signals received from the accelerometer;
sample the filtered signals to generate a power spectrum; and
scale power values in the power spectrum to current values for the at least one stimulation pulse.

12. The implantable pulse generator of claim 8 wherein the computing device is configured to increase a randomness of the at least one stimulation pulse using least significant bits of a device clock of the implantable pulse generator.

13. The implantable pulse generator of claim 8 wherein the computing device is configured to apply charge balancing to limit a total amount of current delivered via the at least one stimulating electrode.

14. A method for applying noise stimulation to a subject, the method comprising:
measuring accelerations using an accelerometer;
generating, using the accelerometer, signals indicative of the measured accelerations; and
generating, using an implantable pulse generator, at least one stimulation pulse based on the generated signals.

15. The method of claim 14 wherein generating at least one stimulation pulse comprises mapping, using a computing device, the signals to at least one output parameters of the at least one stimulation pulse.

16. The method of claim 15 wherein measuring accelerations comprises:
measuring an acceleration in a first direction;
measuring an acceleration in a second direction orthogonal to the first direction; and
measuring an acceleration in a third direction orthogonal to the first and second directions.

17. The method of claim 16 wherein mapping the signals comprises:
mapping the acceleration in the first direction to a frequency of the at least one stimulation pulse;
mapping the acceleration in the second direction to a pulse width of the at least one stimulation pulse; and
mapping the acceleration in the third direction to an amplitude of the at least one stimulation pulse.

18. The method of claim 15 wherein mapping the signals comprises:
filtering the signals;
sampling the filtered signals to generate a power spectrum; and
scaling power values in the power spectrum to current values for the at least one stimulation pulse.

19. The method of claim 14 further comprising increasing a randomness of the at least one stimulation pulse using least significant bits of a device clock of the implantable pulse generator.

20. The method of claim 14 further comprising applying charge balancing to limit a total amount of current delivered via the at least one stimulating electrode.
